(19) United States
(12) Patent Application Publication
(10) Pub. No.: US 2023/0071297 A1
(43) Pub. Date: Mar. 9, 2023

(54) POLARIZED FLUORESCENT LIGHT THERAPY SYSTEM AND METHOD

(71) Applicants: KLOX TECHNOLOGIES INC., Laval (CA); KLOX TECHNOLOGIES DENMARK APS, Ballerup (DK)

(72) Inventors: Mehrnoush DEHGHANI, Laval (CA); Lise HEBERT, Montreal (CA); Michael CANOVA ENGELBRECHT NIELSEN, Frederiksberg C. (DK); Michela ZAGO, Laval (CA); Shannon CAMPBELL, Montreal (CA); Maiken MELLERGAARD, Ballerup (DK); Michael HAMBLIN, Melrose (GB)

(21) Appl. No.: 17/794,663

(22) PCT Filed: Jan. 22, 2021
(86) PCT No.: PCT/CA2021/050069

§ 371 Date: Jul. 22, 2022

Related U.S. Application Data
(60) Provisional application No. 62/964,795, filed on Jan. 23, 2020.

(51) Int. Cl. A61N 5/06

(52) U.S. Cl. CPC A61N 5/062; A61N 2005/0658; A61N 2005/0667; A61N 2005/0651; A61N 2005/0654; A61N 2005/073

(57) ABSTRACT

The present disclosure generally relates to fluorescent light energy system and to methods of using such systems in the modulation of inflammatory response, in particular in inflammatory response associated with healing, and for modulation of a treatment regimen for promoting healing of a wound or a condition of the skin and/or soft tissue.

### CROSS-REFERENCE TO RELATED APPLICATION

This application claims the benefit of and priority to U.S. provisional patent application No. 62/964,795, filed on Jan. 23, 2020; the content of which is herein incorporated in entirety by reference.

### FIELD OF TECHNOLOGY

The present disclosure generally relates to fluorescent light energy system and to methods of using such systems in the modulation of inflammatory response, in particular in inflammatory response associated with healing, and for modulation of a treatment regimen for promoting healing of, for example, a wound or a condition of the skin and/or soft tissue.

### BACKGROUND INFORMATION

Inflammation is part of the complex biological response of body tissues to harmful stimuli, such as pathogens, damaged cells, or irritants, and is a protective response involving immune cells, blood vessels, and molecular mediators. The function of inflammation is to eliminate the initial cause of cell injury, clear out necrotic cells and tissues damaged from the original insult and the inflammatory process, and initiate tissue repair.

Wound healing is an important physiological process that regenerates skin integrity after trauma resulting from either accident or intent procedure. When trauma occurs, a sequential cascade of molecular and cellular events is triggered to initiate tissue repair processes and regeneration. These events involve the three phases of wound healing: hemostasis and inflammatory reaction, cell proliferation, and tissue remodeling. These stages are not mutually exclusive, but rather overlap through time.

Photobiomodulation (PBM) has been considered as a possible therapy for wound management. PBM describes the use of visible light to stimulate biological functions in a non-thermal and non-cytotoxic manner. Studies have demonstrated that PBM reduces inflammation and stimulates healing and tissue repair. Advances in understanding how PBM achieves its biological impact have identified endogenous chromophores that are widely expressed in different cell types, including skin cells, as well as in the extracellular matrix. Specifically, PBM has been demonstrated to directly activate endogenous chromophores (also known as non-visual photoreceptors) including: cytochrome C oxidase (CCO), a small hemeprotein that is associated with the mitochondria inner membrane and that is sensitive to red and NIR (˜610-950 nm) light; flavins, a family of cryptochrome proteins that are involved in the repair of DNA and that are sensitive to blue (˜410-500 nm) light; opsins, a family of proteins that are able to modulate calcium channels, thereby impacting intra-cellular calcium levels and that respond to blue and green (˜410-550 nm) light.

Fluorescent light energy (FLE) is a form of PBM and has been demonstrated to influence inflammatory processes. Studies have demonstrated that acute incisional wounds have reduced inflammation, as well as more physiologic re-epithelization and collagen remodeling, resulting in better quality and less visible scars.

Polarized light in particular has been utilized for a number of clinical applications including wound healing. Although its exact mechanism of action remains unknown, its advantageous effects over scattered light could be due to its higher capacity to penetrate skin, and thus to reach deeper tissues involved in wound healing. Some in vitro studies indicate that polarized light increases fibroblast proliferation and expression of type 1 procollagen, which is essential for the process of wound healing. Furthermore, biological tissues, such as the cornea and the skin, contain near-order light scatterers such as collagen fibrils that could also affect polarization properties of the penetrating light.

In light of this, there remains a need in the field of photobiomodulation to provide a more specific and targeted mean of modulating inflammatory response in order to more efficiently treat biological processes influenced by inflammation such as, for example, healing of skin conditions and disorders.

### SUMMARY OF TECHNOLOGY

According to various aspects, the present disclosure relates to a fluorescent light energy system comprising: a biophotonic composition; and at least one light polarizing element. The biophotonic composition and the at least one polarizing element are in operative alignment such that a fluorescence light energy emitted by the photoactivated biophotonic composition is polarized by the light polarizing element resulting in a polarized fluorescent light energy. In some implementations of these aspects, the biophotonic composition is photoactivated by a light source. In some further implementations, the biophotonic composition comprises one or more light-absorbing molecule.

According to various aspects, the present disclosure relates to a method for modulating an inflammatory response in a tissue, the method comprising: photoactivating a biophotonic composition to cause the biophotonic composition to emit fluorescence; polarizing the fluorescence emitted by the photoactivated biophotonic composition to obtain a polarized fluorescent light energy; and exposing the tissue to the polarized fluorescent light energy. Exposure of the tissue to the polarized fluorescent light energy modulates the inflammatory response in the tissue.

According to various aspects, the present disclosure relates to a method for modulating an immune response in a tissue, the method comprising: photoactivating a biophotonic composition to cause the biophotonic composition to emit fluorescence; polarizing the fluorescence emitted by the photoactivated biophotonic composition to obtain a polarized fluorescent light energy; and exposing the tissue to the polarized fluorescent light energy. Exposure of the tissue to the polarized fluorescent light energy modulates the immune response in the tissue.

According to various aspects, the present disclosure relates to a method for stimulating a quiescent wound healing process, the method comprising: photoactivating a biophotonic composition to cause the biophotonic composition to emit fluorescence; polarizing the fluorescence emitted by the photoactivated biophotonic composition to obtain a polarized fluorescent light energy; and exposing the quiescent wound to the polarized fluorescent light energy. Exposure of the quiescent wound to the polarized fluorescent light energy stimulates healing of the quiescent wound.

According to various aspects, the present disclosure relates to a method for modulating mitochondria biogenesis in a cell population, the method comprising: photoactivating a biophotonic composition to cause the biophotonic composition to emit fluorescence; polarizing the fluorescence emitted by the photoactivated biophotonic composition to obtain a polarized fluorescent light energy; and exposing the tissue to the polarized fluorescent light energy. Exposure of the tissue to the polarized fluorescent light energy modulates mitochondria biogenesis in the tissue.

According to various aspects, the present disclosure relates to the use of polarized fluorescent light energy for modulating an inflammatory response in a tissue.

According to various aspects, the present disclosure relates to the use of polarized fluorescent light energy for modulating an immune response in a tissue.

According to various aspects, the present disclosure relates to the use of polarized fluorescent energy for stimulating a quiescent wound healing process in a subject.

According to various aspects, the present disclosure relates to the use of polarized fluorescent energy for modulating mitochondria biogenesis in a cell population.

Other aspects and features of the present technology will become apparent to those ordinarily skilled in the art upon review of the following description of specific embodiments in conjunction with the accompanying drawings.

### BRIEF DESCRIPTION OF THE DRAWINGS

All features of embodiments which are described in this disclosure are not mutually exclusive and can be combined with one another. For example, elements of one embodiment can be utilized in the other embodiments without further mention. A detailed description of specific embodiments is provided herein below with reference to the accompanying drawings in which:

FIG. 1 shows a diagram outlining an experimental setup to assess the effect of polarized light generated by an embodiment of the present technology.

FIG. 2 shows graphs illustrating the light spectra from a multi-LED lamp, FLE and FLE+filter. Panel A: The spectra from the LED alone (black) or with polarizing filters (linear or circular: green or red, respectively). Panel B: The spectra from FLE alone or filtered by linear or circular polarizers. Panel C: The spectra from FLE alone or filtered by circular polarizing filter with and without adjustment of FLE power intensity to deliver the same fluence. Power outlet (adjusted samples) was increased by 43% for the circular filter. Panel D: The spectra from FLE alone or filtered by linear polarizing filter with and without adjustment of FLE power intensity to deliver the same fluence. Power outlet (adjusted samples) were increased by 63% for the circular filter.

FIG. 3 shows the effects of polarity on immune response (IL-6 expression from inflamed HDFs). Panel A: The application of a right-handed circularly polarizing filter significantly decreases the effect of FLE on IL-6 production measured 24 hours after illumination. Panel B: Similarly, a linearly polarizing filter significantly reduces FLE impact on IL-6 secretion from HDFs as measured 6 and 24 hours post illumination, while linear polarization of LED emitted blue light does not alter its biological effects. *P<0.05, **P<0.005, and ***P<0.0005, N=3. HDF cells were inflammatory stimulated using a cocktail of IL1-α, IL1-β. CTRL-ST: control stimulated.

FIG. 4 shows a graph indicating that FLE treatment down-regulates myeloid cell surface markers in human skin. Ex-vivo human skin punches were treated with non-polarized LED (lamp minus gel), non-polarized FLE (lamp plus gel), or prepared directly after excision without treatment (˜). Transcriptional regulation was analysed in total RNA (from full thickness skin samples) 24 h post illumination.

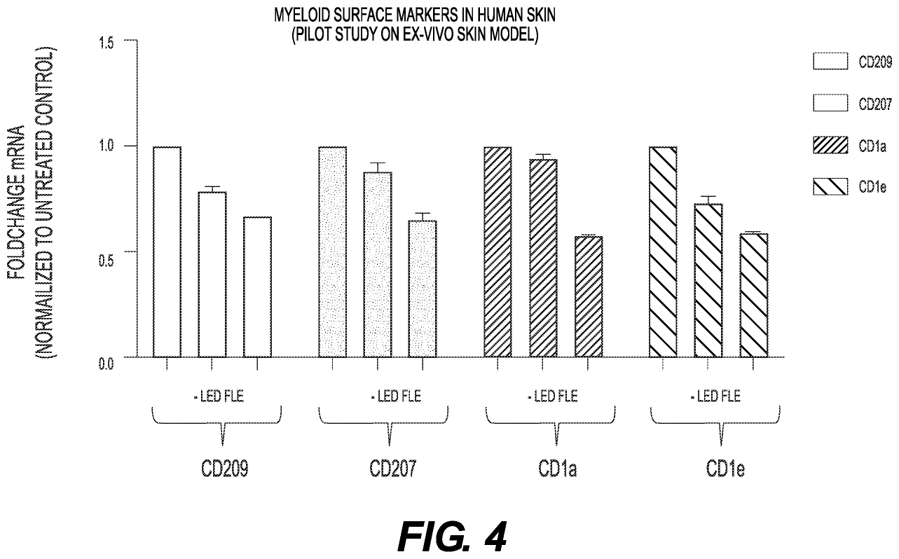

### DETAILED DISCLOSURE

The present technology is explained in greater detail below. This description is not intended to be a detailed catalog of all the different ways in which the technology may be implemented, or all the features that may be added to the instant technology. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which variations and additions do not depart from the present technology. Hence, the following description is intended to illustrate some particular embodiments of the technology, and not to exhaustively specify all permutations, combinations and variations thereof.

As used herein, the singular form “a,” “an” and “the” include plural referents unless the context clearly dictates otherwise.

The recitation herein of numerical ranges by endpoints is intended to include all numbers subsumed within that range (e.g., a recitation of 1 to 5 includes 1, 1.5, 2, 2.75, 3, 3.80, 4, 4.32, and 5).

The term “about” is used herein explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. For example, the term “about” in the context of a given value or range refers to a value or range that is within 20%, preferably within 15%, more preferably within 10%, more preferably within 9%, more preferably within 8%, more preferably within 7%, more preferably within 6%, and more preferably within 5% of the given value or range.

The expression “and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

The term “biophotonic” as used herein refers to the generation, manipulation, detection and application of photons in a biologically relevant context. As used herein, the expression “biophotonic composition” refers to a composition as described herein that may be activated by light to produce photons for biologically relevant applications. As used herein, the expression “biophotonic regimen” or “biophotonic treatment” or “biophotonic therapy” or “FLE-based treatment” refers to the use of a combination of a biophotonic composition as defined herein and an illumination period of that biophotonic composition to activate the biophotonic composition.

Terms and expressions “light-absorbing molecule”, “light-capturing molecule”, “photoactivating agent”, “chromophore” and “photoactivator” are used herein interchangeably. A light-absorbing molecule means a molecule or a complex of molecules, which when contacted by light irradiation is capable of absorbing the light. The light-absorbing molecules readily undergo photoexcitation and, in some instances, can then transfer its energy to other molecules or emit it as light.

The expression “actinic light” as used herein refers to light energy emitted from a specific light source (e.g., lamp, LED, or laser) and capable of being absorbed by matter (e.g., the light-absorbing molecule defined above). In some embodiments, the actinic light is visible light or light in the spectrum of the visible range of wavelength.

As used herein, the term “treated” in expressions such as: “treated skin” and “treated area/portion of the skin” and “treated soft tissue”, refers to a skin or soft tissue surface or layer(s) onto which a method according to the embodiments of the present disclosure has been performed. For example, in some instances, a treated skin or soft tissue refers to a skin onto which the composition of the present disclosure has been applied and which has been illuminated as outlined herein.

As used herein, the term “polarized” used in the context of expressions such as: “polarized light” or “polarized FLE” refers to waves (e.g., light waves) in which the vibrations occur in a single phase. The process of transforming unpolarized wave (e.g., unpolarized light) into polarized waves (e.g., polarized light) is referred to herein as “polarization”.

In some aspects of these embodiments, the expression “biological tissue” refers to any organ and tissue of a living system or organism. Examples of biological tissue include, but are not limited to: brain, the cerebellum, the spinal cord, the nerves, blood, lungs, heart, blood vessels, skin, hair, fat, nails, bones, cartilage, ligaments, tendons, ovaries, fallopian tubes, uterus, vagina, mammary glands, testes, vas deferens, seminal vesicles, prostate, salivary glands, esophagus, stomach, liver, gallbladder, pancreas, intestines, rectum, anus, kidneys, ureters, bladder, urethra, the pharynx, larynx, bronchi, lungs, diaphragm, hypothalamus, pituitary gland, pineal body or pineal gland, thyroid, parathyroid, adrenals (e.g., adrenal glands), lymph nodes and vessels, skeletal muscles, smooth muscles, cardiac muscle, brain, spinal cord, peripheral nervous system, ears, eyes, nose, and the like. In other aspects of these embodiments, the expression “biological tissue” refers to individual cells or a population or a group of cells. In some instances, the cells are ex vivo cells. In some other instances, the cells are in vivo. In some other instances, the cells are in vitro.

As used herein, the expression “cellular processes” refers to processes that are carried out at the cellular level but are not necessarily restricted to a single cell. For example, cell communication occurs among more than one cell, but occurs at the cellular level.

Results from in vivo and in vitro studies have suggested a potential ability of FLE to modulate inflammation associated with down-regulation of pro-inflammatory cytokines such as IL-6 and TNF-α, and stimulation of mitochondria biogenesis. A recent study has suggested that FLE-stimulated cells responded more potently compared to cells treated with light from an LED light source (a “Mimicking Lamp”) designed to generate the same emission spectra and power intensity profile as FLE. FLE-treated human dermal fibroblasts (HDF) experienced up-regulated collagen production, while a minor and non-significant effect was observed for the Mimicking Lamp-treated HDFs. These results suggest that photons generated by FLE either penetrate tissue differently or are absorbed differently compared to photons from an LED light source.

Preliminary study suggests that delivery of FLE to the tissue generates more beneficial cellular processes compared with cells and tissues directly illuminated with a light source alone, or a direct light source with spectra that matches the FLE spectra, resulting in higher cell viability and less inflammation. FLE photons appear to induce a different impact on healing compared with traditional PBM. The effects of light on tissue are due to various degrees of absorption of electromagnetic radiation; however, different characteristics of the radiant fluence delivered to the tissue could impact the degree of absorption and therefore the biological impact. Various light related parameters such as power density, monochromatism, coherence, and polarization may impact penetration or absorption of photons.

Without wishing to be bound to any specific theory, embodiments of the present technology have arisen from a realization that polarized FLE may have effects on biological systems, which effects may be different than those of FLE that is non-polarized. This realization came about by investigating the influence of FLE's polarity on biological systems such as for example on immune parameters such as IL-6 production from inflamed HDFs. It was observed that the biological impact of FLE on the inflammatory phase of wound healing is partly associated with its polarization properties.

In some embodiments, the present technology provides for a fluorescent light energy system (“FLE system”). The FLE system comprises a biophotonic composition and at least one light polarizing element.

In some implementations, the biophotonic composition comprises one or more light-absorbing molecule.

In some implementations, the light polarizing element polarizes fluorescence light emitted by a photoactivated biophotonic composition.

In some implementations, the light polarizing element polarizes FLE.

In some implementations, the light polarizing elements of the present technology are fluorescence polarization elements (e.g., fluorescence polarization filters). The light polarizing elements that may be used in the FLE system of the present technology include, but are not limited to, polarizing filters. In some instances, the polarizing filter is a linear polarizing filter. In some other instances, the polarizing filter is a circular polarizing filter. Circular polarizing filters consist of a linear polarizer on the front, with a quarter-wave plate on the back.

In some embodiments, the FLE system of the present technology uses light emitted from an actinic light source to photoactivate the biophotonic composition. In turn, the photoactivated biophotonic composition emits fluorescence which is then polarized by the light polarizing element giving rise to a polarized FLE. The FLE system of the present technology thus provides for a polarized FLE. The polarized FLE may then be used in various methods which will be discussed in further details herein below.

Biophotonic compositions according to the present disclosure are, in a broad sense, activated by light (e.g., photons) of a specific wavelength. The biophotonic compositions comprise at least one exogenous light-absorbing molecule, which is activated by light and accelerates the dispersion of light energy, which leads to light carrying on a therapeutic effect on its own, and/or to the photochemical activation of other agents contained in the biophotonic composition. When a light-activating molecule absorbs a photon of a certain wavelength, it becomes excited. This is an unstable condition and the light-activating molecule tries to return to the ground state, giving away the excess energy. For some light-activating molecules, it is favorable to emit the excess energy as light when transforming back to the ground state. This process is called fluorescence. The peak wavelength of the emitted fluorescence is shifted towards longer wavelengths compared to the absorption wavelengths (i.e., Stokes' shift). The emitted fluorescent energy can then be transferred to the other components of the composition or to a treatment site on to which the composition is topically applied. Differing wavelengths of light may have different and complementary therapeutic effects on tissue.

In certain implementations, the biophotonic compositions of the present disclosure are substantially transparent/translucent and/or have high light transmittance in order to permit light dissipation into and through the composition. In this way, the area of tissue under the composition can be treated both with the fluorescent light emitted by the composition and the light irradiating the composition to activate it, which may benefit from the different therapeutic effects of light having different wavelengths. The % transmittance of the composition can be measured in the range of wavelengths from 250 nm to 800 nm using, for example, a Perkin-Elmer Lambda 9500 series UV-visible spectrophotometer. Alternatively, a Synergy HT spectrophotometer (BioTek Instrument, Inc.) can be used in the range of wavelengths from 380 nm to 900 nm. The values can be normalized for thickness. As stated herein, % transmittance (translucency) is as measured for a 2 mm thick sample at a wavelength of 526 nm. It will be clear that other wavelengths, thickness of the composition or the like can be used.

In some instances, the biophotonic compositions of the present disclosure are for topical uses (i.e., suitable for topical application). The biophotonic composition can be in the form of a solid, a semi-solid, a liquid, a viscous liquid, such as a gel, or are gel-like, and which have a spreadable consistency at room temperature (e.g., about 20-25° C.) prior to illumination. In certain such instances wherein the biophotonic composition has a spreadable consistency, the biophotonic composition can be topically applied to a treatment site at a thickness of from about 0.5 mm to about 3 mm, from about 0.5 mm to about 2.5 mm, or from about 1 mm to about 2 mm. The biophotonic composition can be topically applied to a treatment site at a thickness of about 2 mm or about 1 mm. Spreadable biophotonic compositions can conform to a topography of an application site. This can have advantages over a non-conforming material in that a better and/or more complete illumination of the application site can be achieved and the biophotonic compositions are easy to apply and remove.

In some embodiments, the biophotonic composition has a transparency or translucency that exceeds 15%, 20%, 25%, 30%, 35%, 40%, 45%, 50%, 55%, 60%, 65%, 70%, 75%, 80%, or 85%. In some embodiments, the transparency exceeds 70%, 80%, 85%, 86%, 87%, 88%, 89%, 90%, 91%, 92%, 93%, 94%, 95%, 96%, 97%, 98% or 99%. All transmittance values reported herein are as measured on a 2 mm thick sample using the Synergy HT spectrophotometer at a wavelength of 526 nm.

In some aspects, the biophotonic compositions of the present disclosure comprise at least a first light-absorbing molecule in a medium, wherein the biophotonic composition is substantially resistant to leaching such that a low or negligible amount of the light-absorbing molecule leaches out of the biophotonic composition into for example skin or onto a soft tissue onto which the biophotonic composition is applied. In certain embodiments, this is achieved by the medium comprising a gelling agent which slows or restricts movement or leaching of the light-absorbing molecule.

Suitable light-absorbing molecules can be fluorescent dyes (or stains), although other dye groups or dyes (biological and histological dyes, food colorings, carotenoids, and other dyes) such as light-absorbing molecules of natural origin can also be used. Suitable light-absorbing molecules can be those that are Generally Regarded As Safe (GRAS), although light-absorbing molecules which are not well tolerated by the skin or other tissues can be included in the biophotonic composition as contact with the skin is minimal in use due to the leaching-resistant nature of the biophotonic composition.

In certain embodiments, the biophotonic composition of the present disclosure comprises at least one light-absorbing molecule which undergoes partial or complete photobleaching upon application of light. In some embodiments, the at least one light-absorbing molecule absorbs and/or emits at a wavelength in the range of the visible spectrum, such as at a wavelength of between about 380 nm and about 800 nm, between about 380 nm and about 700 nm, or between about 380 nm and about 600 nm. In other embodiments, the at least one light-absorbing molecule absorbs/or emits at a wavelength of between about 200 nm and about 800 nm, between about 200 nm and about 700 nm, between about 200 nm and about 600 nm or between about 200 nm and about 500 nm. In other embodiments, the at least one light-absorbing molecule absorbs/or emits at a wavelength of between about 200 nm and about 600 nm. In some embodiments, the at least one light-absorbing molecule absorbs/or emits light at a wavelength of between about 200 nm and about 300 nm, between about 250 nm and about 350 nm, between about 300 nm and about 400 nm, between about 350 nm and about 450 nm, between about 400 nm and about 500 nm, between about 400 nm and about 600 nm, between about 450 nm and about 650 nm, between about 600 nm and about 700 nm, between about 650 nm and about 750 nm or between about 700 nm and about 800 nm.

In some instances, the light-absorbing molecule of the biophotonic composition is selected from a xanthene derivative dye, an azo dye, a biological stain, and a carotenoid. In some instances, the at least one light-absorbing molecule is selected from eosin (e.g., eosin B or eosin Y), erythrosine (e.g., erythrosine B), fluorescein, Rose Bengal, and Saffron red powder.

In certain such embodiments, said xanthene derivative dye is chosen from a fluorene dye (e.g., a pyronine dye, such as pyronine Y or pyronine B, or a rhodamine dye, such as rhodamine B, rhodamine G, or rhodamine WT), a fluorone dye (e.g., fluorescein, or fluorescein derivatives, such as phloxine B, rose bengal, merbromine, Eosin Y, Eosin B, or Erythrosine B, i.e., Eosin Y), or a rhodole dye. In certain such embodiments, said azo dye is chosen from methyl violet, neutral red, para red, amaranth, carmoisine, allura red AC, tartrazine, orange G, ponceau 4R, methyl red, and murexide-ammonium purpurate. In certain such embodiments, said biological stain is chosen from safranin 0, basic fuchsin, acid fuschin, 3,3′ dihexylocarbocyanine iodide, carminic acid, and indocyanine green. In certain such embodiments, said carotenoid is chosen from crocetin, a-crocin (S,S-diapo-S,S-carotenoic acid), zeaxanthine, lycopene, alpha-carotene, beta-carotene, bixin, and fucoxanthine. In certain such embodiments, said carotenoid is present in the composition as a mixture is selected from saffron red powder, annatto extract, and brown algae extract.

In some embodiments, the at least one light-absorbing molecule is present in the biophotonic composition of the present technology in an amount of between about 0.001% and 40% by weight of the composition. In some embodiments, the at least one light-absorbing molecule is present in an amount of between about 0.005% and 2%, between about 0.01% and 1%, between about 0.01% and 2%, between about 0.05% and 1%, between about 0.05% and 2%, between about 0.1% and 1%, between about 0.1% and 2%, between about 1% and 5%, about 2.5% and 7.5%, between about 5% and 10%, between about 7.5% and 12.5%, between about 10% and 15%, between about 12.5% and 17.5%, between about 15% and 20%, between about 17.5% and 22.5%, between about 20% and 25%, between about 22.5% and 27.5%, between about 25% and 30%, between about 27.5% and 32.5%, between about 30% and 35%, between about 32.5% and 37.5%, or between about 35% and 40% by weight of the biophotonic composition. In some embodiments, the at least one light-absorbing molecule is present in an amount of at least about 0.2% by weight of the biophotonic composition.

The biophotonic compositions disclosed herein may include at least one additional light-absorbing molecule. Combining light-absorbing molecules may increase photo-absorption by the combined dye molecules and enhance absorption and fluorescence output. This creates multiple possibilities of generating new photosensitive, and/or selective light-absorbing molecule mixtures. When such multi-light-absorbing molecule compositions are illuminated with light, energy transfer can occur between the light-absorbing molecules. This process, known as resonance energy transfer, is a photophysical process through which an excited ‘donor’ light-absorbing molecule (also referred to herein as first light-absorbing molecule) transfers its excitation energy to an ‘acceptor’ light-absorbing molecule (also referred to herein as second light-absorbing molecule).

In some embodiments, the donor, or first, light-absorbing molecule has an emission spectrum that overlaps at least about 80%, about 70%, about 60%, about 50%, about 40%, about 30%, about 20%, or about 10% with an absorption spectrum of the second light-absorbing molecule. In some embodiments, the first light-absorbing molecule has an emission spectrum that overlaps at least about 20% with an absorption spectrum of the second light-absorbing molecule. In some embodiments, the first light-absorbing molecule has an emission spectrum that overlaps at least between about 1% and 10%, between about 5% and 15%, between about 10% and 20%, between about 15% and 25%, between about 20% and 30%, between about 25% and 35%, between about 30% and 40%, between about 35% and 45%, between about 50% and 60%, between about 55% and 65% or between about 60% and 70% with an absorption spectrum of the second light-absorbing molecule.

Percent (%) spectral overlap, as used herein, refers to the % overlap of a donor light-absorbing molecule's emission wavelength range with an acceptor light-absorbing molecule's absorption wavelength range, measured at spectral full width quarter maximum (FWQM).

In some embodiments, the second light-absorbing molecule absorbs at a wavelength in the range of the visible spectrum. In some embodiments, the second light-absorbing molecule has an absorption wavelength that is relatively longer than that of the first light-absorbing molecule within the range of between about 50 nm and 250 nm, between about 25 nm and 150 nm or between about 10 nm and 100 nm.

As discussed above, the application of light to the biophotonic compositions of the present disclosure can result in a cascade of energy transfer between the light-absorbing molecules. In some embodiments, such a cascade of energy transfer provides photons that penetrate the epidermis, dermis and/or mucosa (or even lower) at the target tissue.

In some embodiments, the light-absorbing molecule is selected such that their emitted fluorescent light, on photoactivation, is within one or more of the green, yellow, orange, red and infrared portions of the electromagnetic spectrum, for example having a peak wavelength within the range of about 490 nm to about 800 nm. In some embodiments, the emitted fluorescent light has a power density of between 0.005 mW/cm² to about 10 mW/cm², about 0.5 mW/cm² to about 5 mW/cm².

Further examples of suitable light-absorbing molecules useful in the biophotonic compositions, methods, and uses of the present disclosure include, but are not limited to the following:

Xanthene Derivatives—

The xanthene group comprises three sub-groups: a) the fluorenes; b) fluoroses; and c) the rhodoles, any of which may be suitable for the compositions, methods, and uses of the present disclosure. The fluorenes group comprises the pyronines (e.g., pyronine Y and B) and the rhodamines (e.g., rhodamine B, G and WT). Depending on the concentration used, both pyronines and rhodamines may be toxic and their interaction with light may lead to increased toxicity. Similar effects are known to occur for the rhodole dye group. The fluorone group comprises the fluorescein dye and the fluorescein derivatives. Fluorescein is a fluorophore commonly used in microscopy with an absorption maximum of 494 nm and an emission maximum of 521 nm. The disodium salt of fluorescein is known as D&C Yellow 8. It has very high fluorescence but photodegrades quickly. In the present composition, mixtures of fluorescein with other photoactivators such as indocyanin green and/or saffron red powder will confer increased photoabsorption to these other compounds.

The eosins group comprises Eosin Y (tetrabromofluorescein, acid red 87, D&C Red 22), a chromophore with an absorption maximum of 514-518 nm that stains the cytoplasm of cells, collagen, muscle fibers and red blood cells intensely red; and Eosin B (acid red 91, eosin scarlet, dibromo-dinitrofluorescein), with the same staining characteristics as Eosin Y. Eosin Y and Eosin B are collectively referred to as “Eosin”, and use of the term “Eosin” refers to either Eosin Y, Eosin B or a mixture of both. Eosin Y, Eosin B, or a mixture of both can be used because of their sensitivity to the light spectra used: broad spectrum blue light, blue to green light and green light. In some embodiments, the composition includes in the range of less than about 12% by weight of the total composition of at least one of Eosin B or Eosin Y or a combination thereof. In some embodiments, at least one of Eosin B or Eosin Y or a combination thereof is present from about 0.001% to about 12%, or between about 0.01% and about 1.2%, or from about 0.01% to about 0.5%, or from about 0.01% to about 0.05%, or from about 0.1% to about 0.5%, or from about 0.5% to about 0.8% by weight of the total composition. In some embodiments, at least one of Eosin B or Eosin Y or a combination thereof is present is an amount of at about 0.005% by weight of the total composition. In some embodiments, at least one of Eosin B or Eosin Y or a combination thereof is present is an amount of at about 0.01% by weight of the total composition. In some embodiments, at least one of Eosin B or Eosin Y or a combination thereof is present is an amount of at about 0.02% by weight of the total composition. In some embodiments, at least one of Eosin B or Eosin Y or a combination thereof is present is an amount of at about 0.05% by weight of the total composition. In some embodiments, at least one of Eosin B or Eosin Y or a combination thereof is present is an amount of at about 0.1% by weight of the total composition. In some embodiments, at least one of Eosin B or Eosin Y or a combination thereof is present is an amount of at about 0.2% by weight of the total composition. In some embodiments, at least one of Eosin B or Eosin Y or a combination thereof is present is an amount of at least about 0.2% by weight of the total composition but less than about 1.2% by weight of the total composition. In some embodiments, at least one of Eosin B or Eosin Y or a combination thereof is present is an amount of at least about 0.01% by weight of the total composition but less than about 12% by weight of the total composition.

In some embodiments, the composition includes in the range of less than 12% by weight of the total composition of at least one of Eosin B or Eosin Y or a combination thereof. In some embodiments, at least one of Eosin B or Eosin Y or a combination thereof is present from 0.001% to 12%, or between 0.01% and 1.2%, or from 0.01% to 0.5%, or from 0.1% to 0.5%, or from 0.5% to 0.8%, or from 0.01% to 0.05%, by weight of the total composition. In some embodiments, at least one of Eosin B or Eosin Y or a combination thereof is present is an amount of at 0.005% by weight of the total composition. In some embodiments, at least one of Eosin B or Eosin Y or a combination thereof is present is an amount of at 0.01% by weight of the total composition. In some embodiments, at least one of Eosin B or Eosin Y or a combination thereof is present is an amount of at 0.02% by weight of the total composition. In some embodiments, at least one of Eosin B or Eosin Y or a combination thereof is present is an amount of at 0.05% by weight of the total composition. In some embodiments, at least one of Eosin B or Eosin Y or a combination thereof is present is an amount of at 0.1% by weight of the total composition. In some embodiments, at least one of Eosin B or Eosin Y or a combination thereof is present is an amount of at 0.2% by weight of the total composition. In some embodiments, at least one of Eosin B or Eosin Y or a combination thereof is present is an amount of at least 0.2% by weight of the total composition but less than 1.2% by weight of the total composition. In some embodiments, at least one of Eosin B or Eosin Y or a combination thereof is present is an amount of at least 0.01% by weight of the total composition but less than 12% by weight of the total composition.

In some embodiments, the composition includes Eosin Y as a first light-absorbing molecule. In some embodiments, the composition includes Eosin Y as a first light-absorbing molecule and any one or more of Rose Bengal, Fluorescein, Erythrosin, Phloxine B as a second light-absorbing molecule. In some embodiments, the composition includes the following synergistic combinations: Eosin Y and Fluorescein; Fluorescein and Rose Bengal; Erythrosine in combination with one or more of Eosin Y, Rose Bengal or Fluorescein; or Phloxine B in combination with one or more of Eosin Y, Rose Bengal, Fluorescein and Erythrosine. Other synergistic light-absorbing molecule combinations are also possible. By means of synergistic effects of the light-absorbing molecule combinations in the composition, light-absorbing molecules which cannot normally be activated by an activating light (such as a blue light from an LED) can be activated through energy transfer from the light-absorbing molecules which are activated by the activating light. In this way, the different properties of photoactivated light-absorbing molecules can be harnessed and tailored according to the therapy required.

In some embodiments, the present disclosure provides compositions that comprise at least a first light-absorbing molecule and a gelling agent. A gelling agent may comprise any ingredient suitable for use in a topical composition as described herein. The gelling agent may be an agent capable of forming a cross-linked matrix, including physical and/or chemical cross-links. The gelling agent is preferably biocompatible, and may be biodegradable. In some implementations, the gelling agent is able to form a hydrogel or a hydrocolloid. An appropriate gelling agent is one that can form a viscous liquid or a semisolid. In preferred embodiments, the gelling agent and/or the composition has an appropriate light transmission property. The gelling agent preferably allows activity of the light-absorbing molecule(s). For example, some light-absorbing molecules require a hydrated environment in order to fluoresce. The gelling agent may be able to form a gel by itself or in combination with other ingredients such as water or another gelling agent, or when applied to a treatment site, or when illuminated with light.

In some embodiments, the biophotonic compositions useful in the present technology is as defined in WO2015/184551 and WO2019/232628, both of which are incorporated herein by reference.

The gelling agent according to various embodiments of the present disclosure may include, but not be limited to, polyalkylene oxides, particularly polyethylene glycol and poly(ethylene oxide)-poly(propylene oxide) copolymers, including block and random copolymers; polyols such as glycerol, polyglycerol (particularly highly branched polyglycerol), propylene glycol and trimethylene glycol substituted with one or more polyalkylene oxides, e.g., mono-, di- and tri-polyoxyethylated glycerol, mono- and di-polyoxy-ethylated propylene glycol, and mono- and di-polyoxyethylated trimethylene glycol; polyoxyethylated sorbitol, polyoxyethylated glucose; acrylic acid polymers and analogs and copolymers thereof, such as polyacrylic acid per se, polymethacrylic acid, poly(hydroxyethylmethacrylate), poly(hydroxyethylacrylate), poly(methylalkylsulfoxide methacrylate), poly(methylalkylsulfoxide acrylate) and copolymers of any of the foregoing, and/or with additional acrylate species such as aminoethyl acrylate and mono-2-(acryloxy)-ethyl succinate; polymaleic acid; poly(acrylamides) such as polyacrylamide per se, poly(methacrylamide), poly(dimethylacrylamide), and poly(N-isopropyl-acrylamide); poly(olefinic alcohol)s such as poly(vinyl alcohol); poly(N-vinyl lactams) such as poly(vinyl pyrrolidone), poly(N-vinyl caprolactam), and copolymers thereof, polyoxazolines, including poly(methyloxazoline) and poly(ethyloxazoline); and polyvinylamines.

In some embodiments, the gelling agent comprises a carbomer. Carbomers are synthetic high molecular weight polymer of acrylic acid that are cross-linked with either allylsucrose or allylethers of pentaerythritol having a molecular weight of about 3×10⁶. The gelation mechanism depends on neutralization of the carboxylic acid moiety to form a soluble salt. The polymer is hydrophilic and produces sparkling clear gels when neutralized. Carbomer gels possess good thermal stability in that gel viscosity and yield value are essentially unaffected by temperature. As a topical product, carbomer gels possess optimum rheological properties. The inherent pseudoplastic flow permits immediate recovery of viscosity when shear is terminated and the high yield value and quick break make it ideal for dispensing. Aqueous solution of Carbopol® is acidic in nature due to the presence of free carboxylic acid residues. Neutralization of this solution cross-links and gelatinizes the polymer to form a viscous integral structure of desired viscosity. Carbomers are available as fine white powders which disperse in water to form acidic colloidal suspensions (a 1% dispersion has a pH of approximately 3) of low viscosity. Neutralization of these suspensions using a base, for example sodium, potassium or ammonium hydroxides, low molecular weight amines and alkanolamines, results in the formation of translucent gels. Nicotine salts such as nicotine chloride form stable water-soluble complexes with carbomers at about pH 3.5 and are stabilized at an optimal pH of about 5.6.

In some implementations, the carbomer is Carbopol®. In some embodiments, from about 0.05% to about 10%, about 0.5% to about 5%, or about 1% to about 3% by weight of the total composition of a high molecular weight carbopol can be present as the gelling agent. In some embodiments, the biophotonic composition of the disclosure comprises from about 0.05% to about 10%, about 0.5% to about 5%, or from about 1% to about 3% by weight of the total composition of a high molecular weight carbopol.

In some embodiments, the gelling agent comprises a hygroscopic and/or a hydrophilic material useful for their water attracting properties. The hygroscopic or hydrophilic material may include, but is not limited to, glucosamine, glucosamine sulfate, polysaccharides, cellulose derivatives (hydroxypropyl methylcellulose, hydroxyethyl cellulose, hydroxypropyl cellulose, methylcellulose and the like), noncellulose polysaccharides (galactomannans, guar gum, carob gum, gum arabic, sterculia gum, agar, alginates and the like), glycosaminoglycan, polyvinyl alcohol), poly(2-hydroxyethylmethylacrylate), polyethylene oxide, collagen, chitosan, alginate, a polyacrylonitrile)-based hydrogel, poly(ethylene glycol)/poly(acrylic acid) interpenetrating polymer network hydrogel, polyethylene oxide-polybutylene terephthalate, hyaluronic acid, high-molecular-weight polyacrylic acid, poly(hydroxy ethylmethacrylate), poly(ethylene glycol), tetraethylene glycol diacrylate, polyethylene glycol methacrylate, and poly(methyl acrylate-co-hydroxyethyl acrylate). In some embodiments, the hydrophilic gelling agent is selected from glucose, modified starch, methyl cellulose, carboxymethyl cellulose, propyl cellulose, hydroxypropyl cellulose, carbomers, alginic acid, sodium alginate, potassium alginate, ammonium alginate, calcium alginate, agar, carrageenan, locust bean gum, pectin, and gelatin.

The gelling agent may be protein-based/naturally derived material such as sodium hyaluronate, gelatin or collagen, lipids, or the like. The gelling agent may be a polysaccharide such as starch, chitosan, chitin, agarose, agar, locust bean gum, carrageenan, gellan gum, pectin, alginate, xanthan, guar gum, and the like.

In some embodiments, the composition can include up to about 2% by weight of the final composition of sodium hyaluronate as the single gelling agent. In some embodiments, the composition can include more than about 4% or more than about 5% by weight of the total composition of gelatin as the single gelling agent. In some embodiments, the composition can include up to about 10% or up to about 8% starch as the single gelling agent. In some embodiments, the composition can include more than about 5% or more than about 10% by weight of the total composition of collagen as the gelling agent. In some embodiments, about 0.1% to about 10% or about 0.5% to about 3% by weight of the total composition of chitin can be used as the gelling agent. In some embodiments, about 0.5% to about 5% by weight of the final composition of corn starch or about 5% to about 10% by weight of the total composition of corn starch can be used as the gelling agent. In some embodiments, more than about 2.5% by weight of the total composition of alginate can be used in the composition as the gelling agent. In some embodiments, the percentages by weight percent of the final composition of the gelling agents can be as follows: cellulose gel (from about 0.3% to about 2.0%), konjac gum (from about 0.5% to about 0.7%), carrageenan gum (from about 0.02% to about 2.0%), xanthan gum (from about 0.01% to about 2.0%), acacia gum (from about 3% to about 30%), agar (from about 0.04% to about 1.2%), guar gum (from about 0.1% to about 1%), locust bean gum (from about 0.15% to about 0.75%), pectin (from about 0.1% to about 0.6%), tara gum (from about 0.1% to about 1.0%), polyvinylypyrrolidone (from about 1% to about 5%), sodium polyacrylate (from about 1% to about 10%). Other gelling agents can be used in amounts sufficient to gel the composition or to sufficiently thicken the composition. It will be appreciated that lower amounts of the above gelling agents may be used in the presence of another gelling agent or a thickener.

In the biophotonic compositions and methods of the present disclosure, additional components may optionally be included, or used in combination with the compositions as described herein. Such additional components include, but are not limited to, chelating agents, polyols, healing factors, growth factors, antimicrobials, wrinkle fillers (e.g. botox, hyaluronic acid or polylactic acid), collagens, anti-virals, anti-fungals, antibiotics, drugs, and/or agents that promote collagen synthesis. These additional components may be applied to the wound, skin or mucosa in a topical fashion, prior to, at the same time of, and/or after topical application of the composition of the present disclosure, and may also be systemically administered. Suitable healing factors, antimicrobials, collagens, and/or agents that promote collagen synthesis are discussed below:

Healing factors comprise compounds that promote or enhance the healing or regenerative process of the tissues on the application site of the composition. During the photoactivation of the composition of the present disclosure, there may be an increase of the absorption of molecules at the treatment site by the skin, wound or the mucosa. An augmentation in the blood flow at the site of treatment is observed for a period of time. An increase in the lymphatic drainage and a possible change in the osmotic equilibrium due to the dynamic interaction of the free radical cascades can be enhanced or even fortified with the inclusion of healing factors. Suitable healing factors include, but are not limited to: hyaluronic acid, glucosamine, allantoin, saffron.

In some embodiments, the biophotonic composition of the present disclosure may be in the form of a membrane. Examples of biophotonic membranes have been described in for example: WO 2019/041032, the entirety of which is incorporated herein by reference.

In some embodiments, the biophotonic composition of the present disclosure may be in the form of fibers or fabrics. Examples of biophotonic fibers or fabrics have been described in for example: WO 2016/065488, the entirety of which is incorporated herein by reference.

In some embodiments of the present technology, the FLE system of the present disclosure may be used in methods for modulating an inflammatory process in a cell population or in a tissue. In some implementations of these embodiments, the modulation is a stimulation of an inflammatory process in the cell population or in the tissue.

In some embodiments of the present technology, the FLE system of the present disclosure may be used in methods for adjustment of an inflammatory process in a cell population or in a tissue. In some implementations of these embodiments, the adjustment of the inflammatory process is a stimulation of an inflammatory process in the cell population or in the tissue.

In some embodiments of the present technology, the FLE system of the present disclosure may be used in methods for modulating an immune response in a tissue. In some implementations of these embodiments, the modulation is stimulation of an immune response in the tissue.

In some implementations of the embodiments of the present disclosure, the FLE system of the present disclosure may be used in a method for modulation of mitochondria biogenesis in a cell population.

In these embodiments, the methods comprise applying a biophotonic composition of the present disclosure to a tissue or a cell population in need of photobiomodulation (PBM) and illuminating the applied composition with actinic light having a wavelength that overlaps with an absorption spectrum of the at least one light-absorbing molecule of the composition so as to photoactivate the light-absorbing molecule causing it to emit FLE. The method further comprises polarizing the FLE emitted with a polarizing element and illuminating the tissue or cell population in need of photobiomodulation with the polarized FLE for a time sufficient to achieve PBM.

In the methods of the present disclosure, any source of actinic light can be used to illuminate or photoactivate the biophotonic composition. Any type of halogen, LED or plasma arc lamp or laser may be suitable. The primary characteristic of suitable sources of actinic light will be that they emit light in a wavelength (or wavelengths) appropriate for activating the one or more photoactivators present in the composition. In some instances, an argon laser is used. In some instances, a potassium-titanyl phosphate (KTP) laser (e.g., a GreenLight™ laser) is used. In other instances, sunlight may be used. In some instances, a LED photocuring device is the source of the actinic light. The source of the actinic light is a source of light having a wavelength between about 200 nm and about 800 nm, between about 400 nm and about 700 nm, between about 400 nm and about 600 nm, between about 400 nm and about 550 nm, between about 380 nm and about 700 nm, between about 380 nm and about 600 nm, between about 380 nm and about 550 nm, between about 200 nm and about 800 nm, between about 400 nm and about 700 nm, between about 400 nm and about 600 nm, between about 400 nm and about 550 nm, between about 380 nm and about 700 nm, between about 380 nm and about 600 nm, or between about 380 nm and about 550 nm. In some instances, the composition of the disclosure is illuminated with violet and/or blue light. Furthermore, the source of actinic light should have a suitable power density. Suitable power density for non-collimated light sources (LED, halogen or plasma lamps) are in the range from about 1 mW/cm² to about 1200 mW/cm², such as from about 20 mW/cm² to about 1000 mW/cm² from about 100 mW/cm² to about 900 mW/cm² from about 200 mW/cm² to about 800 mW/cm², or from about 1 mW/cm² to about 200 mW/cm². In some embodiments, the power density for non-collimated light sources (LED, halogen or plasma lamps) are in the range from about 1 mW/cm² to about 200 mW/cm² Suitable power density for laser light sources is in the range from about 0.5 mW/cm² to about 0.8 mW/cm².

In some embodiments of the methods of the present disclosure, the light has an energy at the subject's skin of from about 1 mW/cm² to about 500 mW/cm², or about 1 mW/cm² to about 300 mW/cm², or about 1 mW/cm² to about 200 mW/cm², wherein the energy applied depends at least on the condition being treated, the wavelength of the light, the distance of the subject's skin from the light source, and the thickness of the composition. In some embodiments, the light at the subject's skin is from about 1 mW/cm² to about 40 mW/cm², or about 20 mW/cm² to about 60 mW/cm², or about 40 mW/cm² to about 80 mW/cm², or about 60 mW/cm² to about 100 mW/cm², or about 80 mW/cm² to about 120 mW/cm², or about 100 mW/cm² to about 140 mW/cm², or about 120 mW/cm² to about 160 mW/cm², or about 140 mW/cm² to about 180 mW/cm², or about 160 mW/cm² to about 200 mW/cm², or about 110 mW/cm² to about 240 mW/cm², or about 110 mW/cm² to about 150 mW/cm², or about 190 mW/cm² to about 240 mW/cm².

In some embodiments, the time of exposure of the tissue or skin to polarized FLE emitted by the photoactivated biophotonic composition is a period from about 1 second to about 30 minutes, from about 1 minute to about 30 minutes, from about 1 minute to about 5 minutes, from about 1 minute to about 5 minutes, from about 20 seconds to about 5 minutes, from about 60 seconds to about 5 minutes, or for less than about 5 minutes, or between about 20 seconds to about 5 minutes, or from about 60 seconds to about 5 minutes per cm² of the area to be treated, so that the total time of exposure of a 10 cm² area would be from about 10 minutes to about 50 minutes.

In some embodiments, the biophotonic composition is illuminated for a period from about 1 minute and 3 minutes. In some embodiments, light is applied for a period of from about 1 second to about seconds, from about 1 second to about 60 seconds, from about 15 seconds to about 45 seconds, from about 30 seconds to about 60 seconds, from about 0.75 minute to about 1.5 minutes, from about 1 minute to about 2 minutes, from about 1.5 minutes to about 2.5 minutes, from about 2 minutes to about 3 minutes, from about 2.5 minutes to about 3.5 minutes, from about 3 minutes to about 4 minutes, from about 3.5 minutes to about 4.5 minutes, from about 4 minutes to about 5 minutes, from about 5 minutes to about 10 minutes, from about 10 minutes to about 15 minutes, from about 15 minutes to about 20 minutes, from about 20 minutes to about 25 minutes, or from about 20 minutes to about 30 minutes. In some embodiments, light is applied for a period of 1 second, about 5 seconds, about 10 seconds, about 20 seconds, about 30 seconds, less than about 30 minutes, less than about 20 minutes, less than about 15 minutes, less than about 10 minutes, less than about 5 minutes, less than about 1 minute, less than about 30 seconds, less than about seconds, less than 10 seconds, less than 5 seconds, or for less than 1 second.

In some instances, multiple applications of the biophotonic composition and exposure to polarized FLE may be performed. In some instances, the tissue or skin is exposed to polarized FLE at least two, three, four, five or six times. In some embodiments, the tissue or skin is exposed to polarized FLE at least two, three, four, five, ten, 25, 50, 100, 200 times with a resting period in between each exposure or it could be back-to-back exposures. In certain such embodiments, the resting period is less than about 1 minute, less than about 5 minutes, less than about 10 minutes, less than about 20 minutes, less about 40 minutes, less than about 60 minutes, less than about 2 hours, less than about 4 hours, less than about 6 hours, or less than 12 hours. In some embodiments, the entire treatment may be repeated in its entirety as may be required by the patient. In some embodiments, a fresh application of the composition is applied before another exposure to polarized FLE.

The methods of the disclosure may be performed at regular intervals such as once a week. The methods of the disclosure may be performed once per week for one or more weeks, such as once per week for one week. The methods of the disclosure may be performed once per week for two weeks, once per week for three weeks, once per week for four weeks, once per week for five weeks, once per week for six weeks, once per week for seven weeks, or once per week for eight or more weeks.

In other embodiments, the methods of the present disclosure may be performed multiple times once or twice a week.

In some embodiment, the FLE systems and methods of the present disclosure may be used to treat skin conditions.

The FLE systems and methods of the present disclosure may be used to treat non-healing wounds and promote healing or granulation tissue formation. Non-healing wounds that may be treated by the biophotonic compositions and methods of the present disclosure include, for example, those arising from acute wounds, injuries to the skin and subcutaneous tissue initiated in different ways (e.g., pressure ulcers from extended bed rest or from being in a non-ambulatory state or due to a presence (whether repeated or chronic) of an external factor such as a therapeutic device such as a cast or a non-therapeutic device such as a saddle or similar device for a non-human animal), wounds induced by trauma, wounds induced by conditions such as periodontitis), and with varying characteristics. In certain embodiments, the present disclosure provides FLE systems and methods for treating and/or promoting the healing of, for example, skin diseases that result in a break of the skin or in a wound, clinically infected wounds, burns, incisions, excisions, lacerations, abrasions, puncture or penetrating wounds, gun-shot wounds, surgical wounds, contusions, hematomas, crushing injuries, sores and ulcers.

FLE systems and methods of the present disclosure may be used to treat and/or promote the healing of chronic cutaneous ulcers or wounds, which are wounds that have failed to proceed through an orderly and timely series of events to produce a durable structural, functional, and cosmetic closure. The vast majority of chronic wounds can be classified into three categories based on their etiology: pressure ulcers, neuropathic (diabetic foot) ulcers and vascular (venous or arterial) ulcers.

In certain other embodiments, the present disclosure provides FLE systems and methods for treating and/or promoting healing, Grade I-IV ulcers. In certain embodiments, the application provides compositions suitable for use with Grade II and Grade III ulcers in particular. Ulcers may be classified into one of four grades depending on the depth of the wound: i) Grade I: wounds limited to the epithelium; ii) Grade II: wounds extending into the dermis; iii) Grade III: wounds extending into the subcutaneous tissue; and iv) Grade IV (or full-thickness wounds): wounds wherein bones are exposed (e.g., a bony pressure point such as the greater trochanter or the sacrum).

The FLE systems and methods of the present disclosure may be used to treat non-healing wounds and promote stimulation of quiescent wound healing. As used herein, the expression “quiescent wound” refers to a wound for which the healing process has stalled or has significantly slowed down.

The present disclosure also provides kits for preparing a FLE system of the present disclosure. In some embodiments, the kit includes one or more light polarizing element as defined herein; and a biophotonic composition as defined here. In some instances, the kit may further comprise a light source. In certain embodiments of the kit, the kit may further comprise written instructions on how to use the FLE system in accordance with the present disclosure.

Variations and modifications will occur to those of skill in the art after reviewing this disclosure. The disclosed features may be implemented, in any combination and sub-combinations (including multiple dependent combinations and sub-combinations), with one or more other features described herein. The various features described or illustrated above, including any components thereof, may be combined or integrated in other systems. Moreover, certain features may be omitted or not implemented. Examples of changes, substitutions, and alterations are ascertainable by one skilled in the art and could be made without departing from the scope of the information disclosed herein.

All references cited herein are incorporated by reference in their entirety and made part of this application.

Practice of the disclosure will be still more fully understood from the following examples, which are presented herein for illustration only and should not be construed as limiting the disclosure in any way.

### EXAMPLES

The examples below are given to illustrate the practice of various embodiments of the present disclosure. They are not intended to limit or define the entire scope of this disclosure. It should be appreciated that the disclosure is not limited to the particular embodiments described and illustrated herein but includes all modifications and variations falling within the scope of the disclosure as defined in the appended embodiments.

### Example 1—Determining the Properties of FLE: Experimental Set-Up

A FLE system according to one embodiment of the present technology was tested to determine the properties of the emitted fluorescence. The tested FLE system comprised a multi-LED lamp and a biophotonic composition (Eosin Y, 0.30% w/w of final composition) having a thickness of 2 mm. The multi-LED lamp used for the study delivered non-coherent blue light with a single peak wavelength between about 440 nm and about 460 nm. The power density was between 110 mW/cm² and 150 mW/cm² at 5 cm from the light source.

A linear polarizing filter or a right-handed circular polarizing filter (Edmund Optics, Barrington, N.J., USA) was applied to the biophotonic composition. As these filters do not fully transmit the light, the LED intensity in the control set (without filter) was adjusted so that the energy level delivered to the cells in all groups was consistent (7.6 J/cm²±5%).

The following equipment was used in the experiment, a detector probe EOP146 was fiber coupled to a CAS140CT array spectroradiometer (from instrument systems), with a wavelength range of 357.8 nm to 832 nm. The light source used was a LED light source, using a blue LED with a peak wavelength of 447.6 nm.

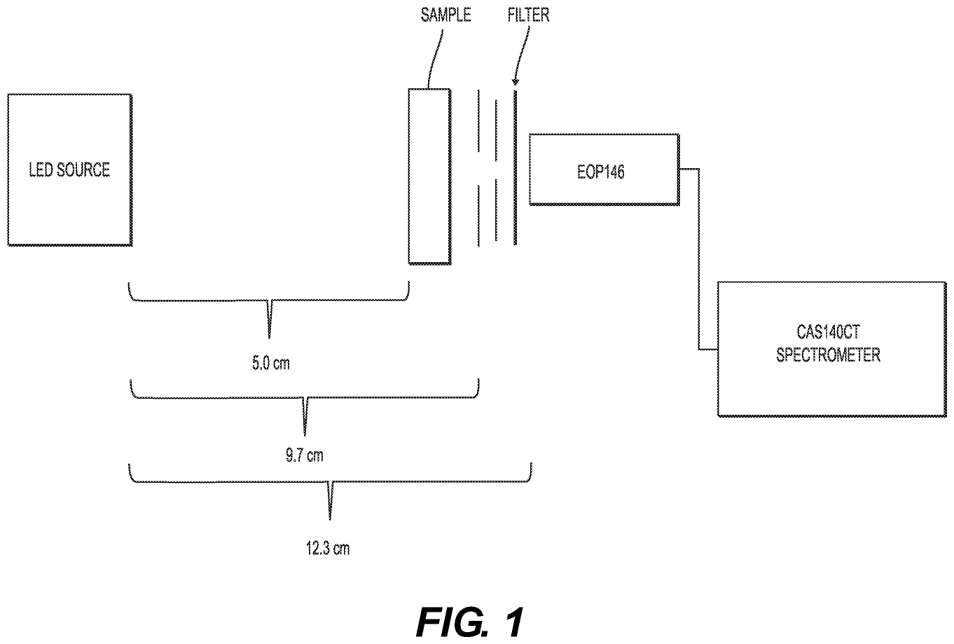

The experimental set-up is illustrated in FIG. 1. The detector probe was placed 12.3 cm away from the LED light source. The light from the source was baffled with two baffles ensuring that the only light hitting the probe was coming from the sample. The filter was placed to the right of the biophotonic composition. The biophotonic composition was placed 5 cm away from the light source. The biophotonic composition comprised a light-absorbing molecule embedded in a gel medium. The biophotonic composition absorbed some of the light from the blue-emitting multi-LED lamp and, through a Stokes shift in the illuminated chromophore, emitted FLE in the range of approximately 510 nm and 700 nm.

Normal dermal human fibroblast (DHFs) cultures were purchased from American Type Culture Collection (ATCC, Manassas, Va., USA) and were cultured at 37° C. and 5% CO₂ in fibroblast basal medium (phenol red-free), supplemented with Fibroblasts Growth Kit-Low serum (ATCC). Culture process was performed seeding DHFs in 12-well plastic plates and incubated for 5-6 hours prior inducing an in vitro inflammatory state with 20 ng/ml of pro-inflammatory cytokines IL-1α/β for 18 hours. The next day, the media was replaced with Phosphate Buffer Saline (PBS) during the illumination procedure and then fresh media containing IL-1α/β cocktail was added to continue with the inflammatory stimulus. The culture supernatant (SN) was collected at 6 hours and 24 hours after illumination with FLE system for cytokine analysis.

Concentrations of secreted IL-6 was measured in collected supernatants using the Quantikine enzyme-linked immunosorbent assay (ELISA) kit in accordance with the manufacturer's protocol (R&D Systems, Minneapolis, Minn., USA). Absorbance at 450 nm was determined using the Synergy HT microplate reader (Biotek, Winooski, Vt., USA) and corrected for absorbance at 570 nm. After collecting cell culture supernatants at 6 hours and 24 hours, a cell viability assay was performed, measuring the mitochondrial metabolic activity (XTT) (Invitrogen, Waltham, Mass. USA). Absorbance at 450 nm was determined using the Synergy HT microplate reader (Biotek, Winooski, Vt., USA) and corrected for absorbance at 570 nm. Affymetrix GeneChip Human Genome Array on ex-vivo human skin: Human ex vivo full-thickness scalp skin organ cultures were prepared by 4-mm punches and cultured. Punches (samples) were divided into three groups with 2 punches per group:

Group 1: Untreated control (no light)

Group 2: LED only

Group 3: LED+Polarization (“FLE”)

Illumination was carried out for 9 minutes. Untreated control sample (Group 1) was prepared immediately, while samples from Groups 2 and 3 were prepared 24 hours after treatment. Half of each punch was stored in 500 μL RNA later, overnight at 4° C. RNA was extracted by RNeasy Mini Kit (Qiagen, Hilden, Germany), samples were sent on ice to Immunology, Frederiksberg, Copenhagen University, and stored at −80° C. until analysis. Samples were analyzed by the Center for Genomic Medicine (Copenhagen University Hospital, Denmark) using a Human Gene 2.0 ST Array (includes lincRNA probes) microRNA expression Arrays (Affymetrix, Santa Clara, Calif., USA). Raw data was analyzed as previously described. The result was expressed as a mean±SD.

Normalized IL-6 levels were graphed as the fold change compared to the inflamed cells that were not illuminated (CTRL-ST). The significance of results was analyzed using the Student's t-test. Differences between the groups were considered as statistically significant at *P<0.05, **P<0.005, and ***P<0.0005. Ex-vivo date is pilot data with limited replicates no statistics have been applied.

### Example 2—Effects of Light Spectra Emitted from FLE System

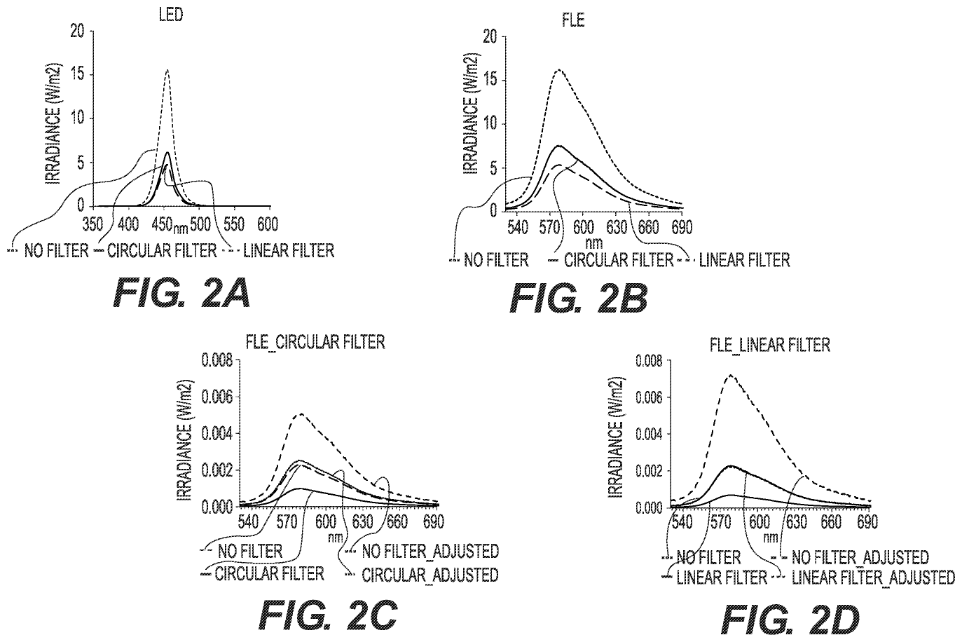

To examine whether light polarity plays a role in FLE's promotion of biological effects, polarizing filters were applied when treating HDFs (i.e., the polarizing filter was placed between the biophotonic composition—as described in Example 1—and the HDFs). The photonic spectra output from multi-LED lamp with or without polarizing filters (FIG. 2, Panel A) and from the illuminated biophotonic composition with or without polarizing filters (FIG. 2, Panel B) were first verified. The measurements indicated a reduction in energy by both filters, which was however, consistent across the entire spectrum, and thus no alterations in specific wavelengths of the spectrum was observed. The loss in energy was adjusted for by increasing power outlet from the lamp (adjusted samples) to ensure the same intensity of the fluence spectra in all treated samples (FIG. 2, Panels C and D).

### Example 3—Effects of Polarity on Immune Response: IL-6 Production

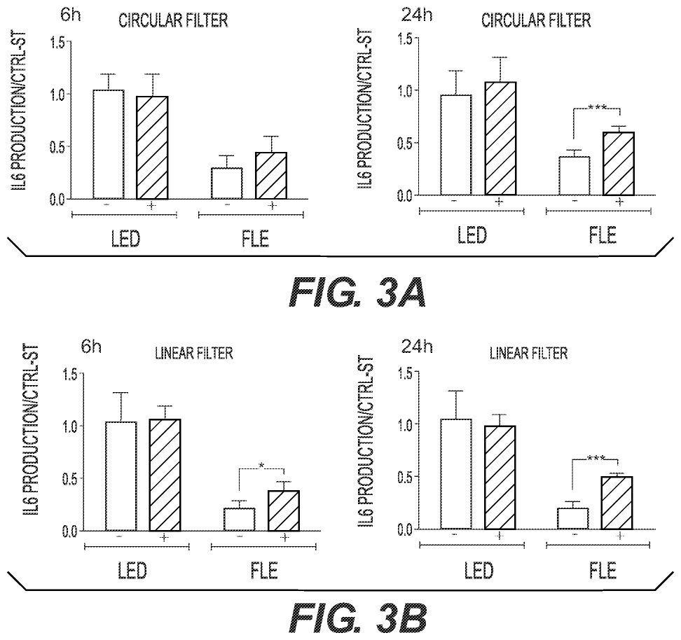

The secreted levels of IL-6 in HDFs at 6 and 24 hours after multi-LED+/−biophotonic composition (as described in Example 1) were compared with or without polarizing filters (FIG. 3). FLE emitted from the biophotonic composition decreased IL-6 secretion by more than 50% within 24 hours compared with LED treatment alone. This inhibitory effect on IL-6 secretion was significantly abrogated at 24 hours in fibroblasts exposed to FLE emitted form the biophotonic composition that had been linearly or circularly polarized.

When compared with FLE irradiated cells without filters, a 63% increase with the circular filter (P=1.4e⁻⁴) was observed, and a 150% increase in IL-6 secretion with the linear filter (P=2.58e⁻⁶) was observed. Thus, the maximum effect of FLE on cellular responses appears to be dependent on the full FLE capacity and availability of non-polarized photons. In addition, polarizing the non-coherent blue light from the multi-LED lamp did not change the fibroblast response when compared to the multi-LED group with no filters.

### Example 4—Myeloid Cellular Markers Found to be Modulated by FLE

The effect of FLE on a human ex-vivo skin model screening for immune modulating factors regulated by FLE was investigated (pilot study). Interestingly, several myeloid immune surface markers were found to be downregulated on the transcriptional level by FLE treatment (FIG. 3). Langerin (CD207) is expressed on Langerhans cells (LCs) residing in the epidermal layer of the skin, whereas CD1 molecules are markers found on both LCs as well as other dermal Dendritic cells (DDCs). CD1 molecules are major histocompatibility complex I (MHC-I) like molecules that present lipids to responding T-cell. CD209 is expressed on dendritic-like macrophages and has been described to facilitate recognition of Cutibacterium acnes (formerly Propionibacterium acnes). These results suggest that FLE treatment target several myeloid cell types and pathways essential for regulating inflammation.

The present study, as outlined in Examples 1 to 4, investigated the role of light polarization on biological effects of FLE, focusing on IL-6 expression from inflamed HDF cells as an inflammatory marker involved in wound healing. The current study, which applies linear and circular polarizing filters, suggests that in addition to their unique wavelength, the photons emitted from the FLE substrate carry other properties that are important for the maximum cellular response. Results highlights that the unique and complete FLE spectrum is critical for the biological impacts, as polarizing non-coherent blue light from the multi-LED lamp alone did not change the response by the HDF cells, and only minor effects were observed on central myeloid surface markers regulated in human skin.

Biological tissues are generally composed of oriented structures, which quickly depolarize transmitted light. However certain tissues and cellular structures such as, eye tissues, superficial skin layers and cell monolayers, which are composed of highly organized bipolar lipid layers in their cell membrane, allow certain degree of light polarization to be transmitted. Several studies report the effect of polarized light on cells and expression of physiologically important biomarkers. The exact mechanism for the unique biological effects of polarized light remains elusive, although the superior penetration capacity of polarized compared to non-polarized light is a plausible hypothesis.

The work presented herein supports that a FLE system positively impacts all phases of healing by modulating the inflammatory cytokines, increasing growth factors and stimulating collagen production.

While the present technology has been described in connection with specific embodiments thereof, it will be understood that it is capable of further modifications and this application is intended to cover any variations, uses, or adaptations of the invention following, in general, the principles of the present technology and including such departures from the present disclosure as come within known or customary practice within the art to which the present technology pertains and as may be applied to the essential features hereinbefore set forth, and as follows in the scope of the appended claims.

1. A fluorescent light energy system comprising:
a biophotonic composition; and
at least one light polarizing element;
wherein the biophotonic composition and the at least one polarizing element are in operative alignment such that a fluorescence light energy emitted by the photoactivated biophotonic composition is polarized by the light polarizing element resulting in a polarized fluorescent light energy.

2. The fluorescent light energy system of claim 1, wherein the biophotonic composition is photoactivated by a light source.

3. The fluorescent light energy system of claim 1 or 2, wherein the biophotonic composition comprises one or more light-absorbing molecule.

4. The fluorescent light energy system of claim 3, wherein the one or more light-absorbing molecule is a xanthene dye.

5. The florescent light energy system of any one of claims 1 to 4, wherein the polarization of the fluorescent light energy is performed with a circular polarizing filter.

6. The florescent light energy system of any one of claims 1 to 4, wherein the polarization of the fluorescent light energy is performed with a linear polarizing filter.

7. A method for modulating an inflammatory response in a tissue, the method comprising:
i) photoactivating a biophotonic composition to cause the biophotonic composition to emit fluorescence;
ii) polarizing the fluorescence emitted by the photoactivated biophotonic composition to obtain a polarized fluorescent light energy; and
iii) exposing the tissue to the polarized fluorescent light energy of step ii),
wherein exposition of the tissue to the polarized fluorescent light energy modulates the inflammatory response in the tissue.

8. The method of claim 7, wherein the modulation is a stimulation of the inflammatory response.

9. The method of claim 7 or 8, wherein the inflammatory response is associated with a tissue infection.

10. The method of claim 9, wherein the tissue infection is a bacterial infection.

11. The method of any one of claims 7 to 9, wherein the inflammatory response is associated with healing.

12. The method of claim 11, wherein the healing is a healing of a tissue.

13. The method of claim 12, wherein the tissue is skin.

14. The method of claim 7, wherein the inflammation response is associated with down-regulation of pro-inflammatory cytokines.

15. The method of claim 14, wherein the pro-inflammatory cytokines include IL-6 and TNF-α.

16. A method for modulating an immune response in a tissue, the method comprising:
i) photoactivating a biophotonic composition to cause the biophotonic composition to emit fluorescence;
ii) polarizing the fluorescence emitted by the photoactivated biophotonic composition to obtain a polarized fluorescent light energy; and
iii) exposing the tissue to the polarized fluorescent light energy of step ii),
wherein exposition of the tissue to the polarized fluorescent light energy modulates the immune response in the tissue.

17. The method of claim 16, wherein the modulation is a stimulation of the immune response.

18. A method for stimulating a quiescent wound healing process, the method comprising:
i) photoactivating a biophotonic composition to cause the biophotonic composition to emit fluorescence;
ii) polarizing the fluorescence emitted by the photoactivated biophotonic composition to obtain a polarized fluorescent light energy; and
iii) exposing the quiescent wound to the polarized fluorescent light energy of step ii),
wherein exposition of the quiescent wound to the polarized fluorescent light energy stimulates healing of the quiescent wound.

19. A method for modulating mitochondria biogenesis in a cell population, the method comprising:
i) photoactivating a biophotonic composition to cause the biophotonic composition to emit fluorescence;
ii) polarizing the fluorescence emitted by the photoactivated biophotonic composition to obtain a polarized fluorescent light energy; and
iii) exposing the tissue to the polarized fluorescent light energy of step ii),
wherein exposition of the tissue to the polarized fluorescent light energy modulates mitochondria biogenesis in the tissue.

20. The method of any one of claims 16 to 19, wherein the biophotonic composition is photoactivated by a light source.

21. The method of any one of claims 16 to 19, wherein the biophotonic composition comprises one or more light-absorbing molecule.

22. The method of claim 22, wherein the one or more light-absorbing molecule is a xanthene dye.

23. The method of any one of claims 16 to 22, wherein the polarization of the fluorescent light energy is performed with a circular polarizing filter.

24. The method of any one of claims 16 to 22, wherein the polarization of the fluorescent light energy is performed with a linear polarizing filter.

25. Use of polarized fluorescent light energy for modulating an inflammatory response in a tissue.

26. Use of polarized fluorescent light energy for modulating an immune response in a tissue.

27. Use of polarized fluorescent energy for stimulating a quiescent wound healing process in a subject.

28. Use of polarized fluorescent energy for modulating mitochondria biogenesis in a cell population.
